(19) United States
(12) Patent Application Publication
(10) Pub. No.: US 2010/0298153 A1
(43) Pub. Date: Nov. 25, 2010

(54) METHODS FOR ANALYSING PROTEIN SAMPLES BASED ON THE IDENTIFICATION OF C-TERMINAL PEPTIDES

(75) Inventor: Ralf Hoffmann, Bruggen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/439,259

(22) PCT Filed: Sep. 3, 2007
(86) PCT No.: PCT/IB2007/053541

§ 371 Date: Feb. 27, 2009

(30) Foreign Application Priority Data
Sep. 14, 2006 (EP) 06120659.5

(51) Int. Cl. C40B 20/08; C07K 1/00; C40B 40/10; C40B 60/10

(52) U.S. Cl. 506/6; 435/272; 506/18; 506/38

(57) ABSTRACT

The present invention relates to a method for identifying proteins in one or more samples based on the isolation and analysis of their C-terminal peptides. The isolated peptides are purified and analysed by Mass spectroscopy. Identification of the parent protein is based on the mass of the C-terminal peptide in combination with additional physicochemical parameters. The present invention further relates to an annotated database of C-terminal peptides of in silico cleaved proteins comprising the masses of C-terminal peptides and one or more physicochemical properties thereof.

### FIELD OF THE INVENTION

The present invention relates to methods for the simultaneous analysis of protein samples using Mass Spectrometry, allowing the selective isolation of peptides from a mixture of cleaved proteins. The present invention further relates to techniques for purifying peptides and data analysis of Mass Spectrometry data.

### BACKGROUND OF THE INVENTION

Different methods have evolved over the last decades to identify proteins using Mass spectrometry (MS). In the so-called fingerprinting method, proteins are isolated and cleaved into peptide fragments. By comparing the mass of the generated peptides with an in silico database of cleaved proteins it is possible to identify the parent protein without further sequence determination.

It is however the aim to study in one single experiment different proteins present in a sample using shotgun approaches. Reducing the complexity of a peptide sample is often performed by selectively isolating internal cysteine-containing peptides. Because cysteine does not appear in every protein, alternative strategies have been developed to specifically isolate N-terminal or C-terminal peptides from the generated peptide mixture. In this way every protein is represented by one peptide. Examples of methods based on the isolation of C-terminal peptides are described e.g. in U.S. Pat. No. 6,156,527 (Schmidt) and US2002/0106700 (Foote). This approach does not allow the classical fingerprinting, which is based on identifying different peptides originating from the same protein. Nevertheless, it would be advantageous to be able to positively identify a peptide (and consequently its parent protein), without having to perform MS/MS peptide sequencing.

U.S. Pat. No. 6,846,679 (Schmidt) discloses a method for selecting C-terminal peptides and comparing the masses of these peptides with a database of C-terminal peptides. The examples of this patent show that for a set of about 1800 C-terminal Lys-C peptides, for only about 45% of the peptides the mass can be unequivocally correlated with a single peptide in the in silico generated database of Lys-C peptides.

US2005/0092910 (Geromanos) discloses a method wherein the mass of a peptide on MS is determined, as well as another physicochemical property of the peptide. This method allows discriminating between peptides having the same mass. However, in view of the fact that complete samples are analysed, numerous different peptides are still generated which have the both same mass and the same physicochemical properties, so that such a peptide cannot be attributed to a single parent protein.

Accordingly there remains a further need for high throughput methods wherein complex protein samples can be analysed by MS without the need for sequencing of the generated peptides on MS/MS.

### SUMMARY OF THE INVENTION

The present invention relates to methods for analysing proteins, including proteins present in complex protein mixtures, based on the cleaving of the proteins and the isolation and analysis of C-terminal peptides therefrom. In the methods of the present invention, isolated C-terminal peptides are subjected to one or more peptide purification steps and to MS analysis. During or after purification, physicochemical properties of the purified peptide other than its mass, are collected. The mass of the purified C-terminal peptides is determined by MS. The peptide is identified based on comparison with a database which combines both mass and one or more physicochemical characteristics of C-terminal peptides. In particular embodiments 2, 3, 4, 5 or up to 10 additional physicochemical characteristics are used to annotate the database. The methods of the present invention allow the positive identification of C-terminal peptides (and their corresponding parent protein, accordingly), with high accuracy without the need for de novo sequence determination on MS/MS.

The advantage of the procedure wherein C-terminal peptides are selected is that each protein is represented by only a single peptide after proteolytic cleavage, leading to a strong reduction in complexity of the sample to be analysed, and the corresponding database containing information of these peptides.

Another advantage of the proposed procedure is that the C-terminal peptides of all proteins are known for organisms for which their genome has been sequenced (such as man, mouse and rat but also lower organisms such as Drosophila, C. elegans and yeasts). The exact molecular weights of these peptides can be predicted, which is expected to support the identification of the peptide underlying a measured mass spec signal. This is particularly true for the currently available high-performance mass spectrometric techniques like FT-ICR, which can achieve resolutions on the order of >500,000 and a mass accuracy of <1 ppm.

In addition, as the nature of expected C-terminal peptides is known from in silico analyses of genomic sequences, a library of synthetic peptides is generated and the exact characteristics of each peptide during the preparation process (e.g., retention time on different chromatographic materials, behaviour in ESI/MALDI-TOF) can be determined and compared to identified peptides from the complex protein mixture. This significantly improves the confidence in correct protein identifications.

It is further an advantage of the present invention that C-terminal peptides stay unmodified in the methods of the invention (apart from alkylation and acetylation which are common modifications in proteomics and do not disturb the down-stream analysis of peptides by mass spectrometry). Interference with ionising processes to evaporate peptides into the gas phase is therefore unlikely.

It is another advantage of the presented method that at least a part of the existing splice variants, namely those occurring at the N-terminal side of proteins can be addressed by the approach of the present invention wherein the less variable C-termini are isolated and identified.

Particular and preferred aspects of the invention are set out in the accompanying independent and dependent claims. Features from the dependent claims may be combined with features of any of the independent claims and with features of other dependent claims as appropriate and not merely as explicitly set out in the claims.

A first aspect of the present invention provides methods for identifying a protein in a protein sample. These methods typically comprise the steps of:
a) modifying carboxyl groups of the proteins in the protein sample,
b) cleaving the proteins in the protein sample into peptides with a cleaving agent,
c) isolating from the cleaved peptides the C-terminal peptides, thereby removing the N-terminal and internal peptides,
d) subjecting the isolated C-terminal peptides to one or more peptide purification steps, so as to obtain purified C-terminal peptides,
e) determining or calculating at least one physicochemical property, other than the mass, of the purified C-terminal peptides,
f) determining the mass of a C-terminal peptides on MS,
g) comparing the mass and the at least one other physicochemical property of the purified C-terminal peptide to a database comprising the mass and one or more physicochemical properties of all C-terminal peptides generated by the cleavage agent, so as to identify the parent protein of the purified C-terminal peptide.

According to one embodiment of the methods of the invention, step (g) comprises identifying for each of the purified C-terminal peptides, one or more C-terminal peptides in the database with a mass corresponding to the purified C-terminal peptide, and, when more than one peptide are identified in the database as corresponding to one purified C-terminal peptide, comparing at least one other physicochemical parameter of the purified C-terminal peptide with those of the more than one peptides identified in the database, so as to positively identify the corresponding C-terminal peptide in the database.

According to one embodiment of the methods of this aspect of the invention, the protein sample is from a species and the database comprises the mass and one or more other physicochemical properties of all C-terminal peptides of that species generated by the cleaving agent.

Particular embodiments of the methods of the present invention, include methods whereby the protein is identified simultaneously in two or more samples and the method accordingly comprises the following additional features:
performing the modification in step (a), with one of a set of differential labelling reagents, different for each of the samples
an additional step of pooling the two or more samples prior to step (d),
identifying prior to step (g) the nature of the label of the isolated peptide so as to identify the sample from which the peptide originates, and
comparing in step (g) the mass and the at least one other physicochemical property of the purified C-terminal peptide to a database comprising the mass of and at least one other physicochemical property of all C-terminal peptides generated by the cleaving agent, so as to identify the C-terminal peptides.

According to a particular embodiment of the methods of the invention, the at least one physicochemical property is determined during the one or more peptide purification steps.

According to a particular embodiment of the methods of the invention, the at least one physicochemical property is selected from the group of pI, retention time during reversed phase chromatography and the ratio of UV absorption at 280 and 214 nm.

In particular embodiments of the methods of the invention, the modification in step (a) is performed using a carbodiimide reaction with primary amines.

In particular embodiments of the methods of the invention, the isolation of C-terminal peptides in step (c) comprises the step of reacting the carboxylgroup of N-terminal and internal peptides via a carbodiimide mediated reaction with a modified biotin carrying a primary amine group.

A further aspect of the present invention provides methods for isolating C-terminal peptides from a protein sample comprising the steps of:
a) reacting carboxyl groups of (intact) proteins in the sample via a carbodiimide with primary amines,
b) cleaving the (intact) proteins with a cleavage agent into peptides,
c) reacting the carboxylgroup of N-terminal and internal peptides via a carbodiimide with an affinity tag carrying a primary amine group.
d) binding the tagged peptides to an affinity matrix and collecting the non-bound peptides (the non-bound peptides being the non-tagged peptides), thereby isolating from the peptides obtained under (b) the C-terminal peptides from the N-terminal and internal peptides.

According to a particular embodiment of the methods of this aspect of the invention, the affinity tag is biotin.

Yet another aspect of the present invention relates to a database of C-terminal peptides of proteins of an organism cleaved in silico by a cleaving agent wherein each peptide is characterised by a protein identifier, the amino acid composition, the mass and one or more other physicochemical properties.

In particular embodiments, the one or more other physicochemical properties of the C-terminal peptides in the database are selected from the group consisting of the calculated retention time on reverse phase chromatography, the net charge at a given pH, and the isoelectric point of the C-terminal peptides.

In a particular embodiments, the database is a database of proteins of a human organism cleaved in silico.

In a further particular embodiment, the database is based on the cleaving of proteins with a cleaving agent which is trypsin.

In a particular embodiment, the peptides in the database include C-terminal peptides resulting from an incomplete cleavage with the cleaving agent whereby one cleavage position is missed.

Yet a further aspect of the present invention relates to the use of a database in the methods described above for the identification of proteins.

Yet a further aspect of the present invention provides a device (100) for identifying proteins in one or more samples based on their C-terminal peptides, the device being characterized in that it comprises at least one sample source (101), a modification/labelling unit (102), with at least one corresponding modifying agent/label source (103), a cleavage unit (104), a C-terminal peptide isolation unit (105), a peptide separation unit (106) with an analysis unit (107) for determining and/or registering one or more physicochemical properties of a purified peptide, a mass spectrometer unit (108) a control circuitry and data analysis unit (109) connected to a read out unit (110). More specifically, the devices of the present invention comprise a connection to a database (111) comprising the masses of all C-terminal peptides of proteins cleaved in silico using a cleaving agent annotated with physicochemical properties of the C-terminal peptides.

### BRIEF DESCRIPTION OF THE DRAWINGS

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference Figures quoted below refer to the attached drawings.

FIG. 1 shows in accordance with a specific embodiment, a method for the isolation of C-terminal peptides. 1: protein denaturation; 2 protein alkylation; 3: protein acetylation; 4: EDC activation of carboxyl groups; 5: reaction of EDC activated carboxyl groups with a primary amine; 6: protein cleavage into N-terminal (a), internal (b) and C-terminal peptides (c); 7: ligation of free carboxyl groups of N-terminal and internal peptides to a purification unit; 8: affinity separation of the C-terminal peptide, which is left in the solution (c).

FIG. 2 shows in accordance with a specific embodiment of the present invention the carbodiimide-mediated reaction between a carboxylgroup on molecule 1 and a primary amine group on molecule 2.

FIG. 3 shows in accordance with a particular embodiment of the present invention, the structure of biotin modified with a primary amine group suitable for carbodiimide mediated reaction with carboxyl groups.

FIG. 4 shows in accordance with a particular embodiment of the present invention a device (100) for isolating and analysing C-terminal peptides of 2 protein samples comprising two sample sources (101), a modification/labelling unit (102), with corresponding modifying agents/label sources (103), a cleavage unit (104), a C-terminal peptide isolation unit (105), a peptide separation unit (106), a mass spectrometer unit (108) and a control circuitry and data analysis unit (109) connected to a read out unit (110). Separation unit (106) comprises two consecutively linked separation systems (1106) and (2106). Mass spectrometer element (108) comprises a unit which separates isotopic forms of peptides. Unit 107 is an analysis unit for determining and/or registering physicochemical properties of peptides purified in (106). Unit 111 is an annotated database of C-terminal peptides. (dotted lines indicate the acquisition of experimental and in silico data).

### DETAILED DESCRIPTION OF THE EMBODIMENTS

In the different Figures, the same reference signs refer to the same or analogous elements.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. Unless specified, these definitions should not be construed to have a scope less than understood by a person of ordinary skill in the art.

The term “polypeptide” or “protein”, as used herein, refers to a plurality of natural or modified amino acids connected via a peptide bond. The length of a polypeptide can vary from 2 to several thousand amino acids (the term thus also includes what is generally referred to as oligopeptides). Included within this scope are polypeptides comprising one or more amino acids which are modified by in vivo posttranslational modifications such as glycosylation, phosphorylation, etc. and/or comprising one or more amino acids which have been modified in vitro with protein modifying agents (e.g. alkylating agents).

The term “polypeptide fragment” or “peptide” as used herein is used to refer to the amino acid sequence obtained after enzymatic cleavage of a protein or polypeptide. A polypeptide fragment or peptide is not limited in size or nature.

The terms “internal”, “N-terminal” and “C-terminal” when referring to a peptide are used herein to refer to the corresponding location of a peptide in a protein or polypeptide. For example, in a tryptic cleavage of protein NH₂—X₁—K—X₂—R—X₃—K—X₄—COOH (wherein X₁, X₂, X₃ and X₄ are peptide sequences of indifferent length without Lysine (K) or Arginine (R)), the N-terminal peptide is NH₂—X₁—K—COOH, the internal peptides are NH₂—X₂—R—COOH and NH₂—X₃—K—COOH and the C-terminal peptide is NH₂—X₄—COOH.

The term “parent protein” refers to the uncleaved protein from which a cleaved peptide is derived.

The term “protein cleavage” as used herein relates to the hydrolysis of a peptide bond between two amino acids in a polypeptide. In the context of physiologic processes, protein cleavage is also referred to as “enzymatic hydrolysis”, “proteolytic processing”, and “protein maturation”. Accordingly, the term “cleaving agent” refers to a compound capable of hydrolysing a peptide bond between two amino acids in a polypeptide or peptide.

The term “fragmentation” as used herein refers to the breaking of one or more chemical bonds and subsequent release of one or more parts of a molecule as obtained e.g. by collision-induced dissociation (CID) in Tandem Mass spectrometry (MS) or MS/MS analysis. In certain embodiments the bond is a peptide bond, but it is not limited thereto.

The term “mass” in the present invention refers to the mass-to-charge ratio (m/z). The abbreviation m/z is used to denote the dimensionless quantity formed by dividing the mass number of an ion by its charge number. The “monoisotopic mass” refers to the mass of the ion containing only the most abundant isotopes. “Average mass” refers to the mass of a particle or molecule of given empirical formula calculated using atomic weights for each element.

The term “label” as used herein refers to a compound or molecule, which can be covalently linked to or incorporated in a peptide or polypeptide and which, based on its particular properties is detectable by optical or other means, such as a Mass Spectrometer. Where the label can be covalently bound to a peptide or polypeptide, this is ensured by a protein/peptide reactive group, present in the labelling reagent. While the term label is generally used in the art, a distinction can be made between the label as such (e.g. as bound to a protein or peptide) and a labelling reagent (the molecule comprising the label prior to the binding with the peptide or protein), capable of binding to a functional group. The present invention envisages the use of different types of labels, such as fluorescent or isotopic labels.

The term “isotopic labels” as used herein refers to a set of labels having the same chemical formula but differing from each other in the number and/or type of isotopes present of one or more atoms, resulting in a difference in mass on MS. Thus, identical peptides labelled with different isotopic labels can be differentiated as such on MS based on a difference in mass.

The term “protein/peptide reactive group” (PRG) as used herein refers to a chemical function on a compound that is capable of reacting with a functional group on an amino acid of a protein or peptide resulting in the binding (non-covalent or covalent) of such compound to the amino acid.

The term “functional group” as used herein refers to a chemical function on an amino acid which can be used for binding (generally, covalent binding) to a chemical compound. Functional groups can be present on the side chain of an amino acid or on the N-terminus or C-terminus of a polypeptide or peptide. The term encompasses both functional groups which are naturally present on a peptide or polypeptide and those introduced via e.g. a chemical reaction using protein-modifying agents.

The present invention describes a method of identifying a parent protein based on the determination of the mass of the corresponding C-terminal peptide and, if necessary, on other physicochemical parameters of this C-terminal peptide.

The methods and tools of the present invention are of particular interest in the analysis of a set of samples for which a simultaneous analysis is of interest. Such a set of samples can be, but is not limited to, samples from a patient taken at different time points, samples of different clinical versions of a disease, samples of different patients etc. The present invention thus provides methods and tools for identifying markers of disease progression, for differential diagnosis, and moreover for multiplex analysis in biochemical or physiological assays.

The methods and tools of the present invention relate to the analysis of protein samples. The term ‘sample’ as used herein is not intended to necessarily include or exclude any processing steps prior to the performing of the methods of the invention. The samples can be rough unprocessed samples, extracted protein fractions, purified protein fractions etc. . . . According to one embodiment the protein samples are pre-processed by immunodepletion of abundant proteins.

Protein samples which are suitable for analysis with the methods of the present invention include samples of viral, prokaryote, bacterial, eukaryote, fungal, yeast, vegetal, invertebrate, vertebrate, mammalian and human origin. Samples can be entire organisms such as homogenates of C. elegans, Drosophila or murine embryo's, or can be tissues or organs of an organism. The preparation of samples differs depending on the organism, tissue or organ investigated, but standard procedures are usually available and known to the expert. With respect to mammalian and human protein samples it covers the isolation of cultured cells, laser micro-dissected cells, body tissue, body fluids, or other relevant samples of interest. With respect to the fractionation of proteins in a sample, cell lysis is the first step in cell fractionation and protein purification. Many techniques are available for the disruption of cells, including physical, enzymatic and detergent-based methods. Historically, physical lysis has been the method of choice for cell disruption; (homogenisation, osmotic lysis, ultrasound cell disruption) however, it often requires expensive, cumbersome equipment and involves protocols that are sometimes difficult to repeat due to variability in the apparatus (such as loose-fitting compared with tight-fitting homogenisation pestles). In recent years, detergent-based lysis has become very popular due to ease of use, low cost and efficient protocols.

Mammalian cells have a plasma membrane, a protein-lipid bilayer that forms a barrier separating cell contents from the extracellular environment. Lipids comprising the plasma membrane are amphipathic, having hydrophilic and hydrophobic moieties that associate spontaneously to form a closed bimolecular sheet. Membrane proteins are embedded in the lipid bilayer, held in place by one or more domains spanning the hydrophobic core. In addition, peripheral proteins bind the inner or outer surface of the bilayer through interactions with integral membrane proteins or with polar lipid head groups. The nature of the lipid and protein content varies with cell type. Clearly, the technique chosen for the disruption of cells, whether physical or detergent-based, must take into consideration the origin of the cells or tissues being examined and the inherent ease or difficulty in disrupting their outer layer(s). In addition, the method must be compatible with the amount of material to be processed and the intended downstream applications.

In particular embodiments, protein extraction also includes the pre-fractionation of cellular proteins originated from different compartments (such as extracellular proteins, membrane proteins, cytosolic proteins, nuclear proteins, mitochondrial proteins). Other pre-fractionation methods separate proteins on physical properties such as isoelectric point, charge and molecular weight.

According to a particular embodiment, the samples are pre-treated prior to modification or cleavage, so as to denature the proteins for optimised access to reagents or proteases, using appropriate agents (e.g., guanidinium chloride, urea, acids (e.g. 0.1% trifluoric acid), bases (e.g. 50% pyridine) and ionic or non-ionic detergents).

The methods of the present invention thus optionally comprise a pre-treatment of the samples, which can be performed in a pre-treatment step comprising one or more of the sample preparation methods listed above. Accordingly, devices suitable for the methods of the present invention optionally comprise a sample preparation unit comprising one or more devices suitable for sample preparation e.g. sonication devices, chromatography systems (affinity, gelfiltration), ultrafiltration units, centrifuges, temperature controlled reaction vials with delivery systems for buffers, enzymes, detergents etc. . . .

The methods of the invention can be applied to one single sample or to two or more samples for comparative analysis, whereby the C-terminal peptides in these samples are provided with a label that can discriminate a same peptide originating from the different samples. Where two or more samples are analysed simultaneously, the pooling of the samples can occur at different time points in the method (as will be detailed below) provided that the pooling occurs after the differential labelling of the individual samples.

In one step of the methods of the present invention, which in most embodiments of the methods of the invention is the first step, the C-termini of the proteins in a sample and the side chains of Asp and Glu, are modified.

Suitable carboxyl modifying agents are, for example, compounds that lead to the formation of carboxylic esters (for example, methanol or other lower aliphatic or alicyclic alcohol, diazomethane, Methyliodide, Me₃SiCHN₂, Me₂C(OMe)₂, CH₃OCH₂Cl, CH₃SCH₂Cl, CH₃OCH₂CH₂OCH₂Cl, PhCH₂OCH₂Cl, Me₃SiCl, Et₃SiCl and Me₂PhSiCl), amides (for example, methylamide, ethylamine, Me₂NH, pyrrolidine, piperidine) and hydrazide derivatives (for example, phenylhydrazine) derivatives. The generation of carboxylic ester derivatives may involve carboxylate activation with a good leaving group followed by displacement with a suitable nucleophile or nucleophile displacement of the carboxylate on an alkyl halide or sulfonate. In certain embodiments, the modifying agent is methyl iodide. In other embodiments, modification of carboxyl groups involves carbodiimide activation (eg with 1-Ethyl-3-[3-dimethylamino-propyl]carbodiimide hydrochloride (EDC)) prior to reaction with a suitable protecting agent. For example, a protecting agent suitable for reaction with a carbodiimide-activated carboxyl group is an aliphatic amine (NH₂—R). In one embodiment, the aliphatic amine is methylamine or ethylamine.

In particular embodiments, also cysteine is modified by e.g. alkylation and/or Lysine is modified by e.g. acetylation. Modification of lysine can be done to modulate the specificity of trypsin or to avoid labelling on the amine group of lysine as explained in detail further on.

In another step of the methods of the present invention, which is generally the step following the above-described modification step, the carboxyl-modified proteins in the sample(s) are cleaved by a cleaving agent.

As detailed below, the final analysis of the samples in the methods of the present invention is performed using Mass Spectrometry (MS). Optimal results are obtained in MS using peptides of up to about 50 amino acids in length. Also for the separation of peptides, most chromatography systems have a higher resolution for peptides than for proteins. Accordingly, the methods of the present invention include a cleavage step, whereby large proteins are reduced to N-terminal, C-terminal and internal peptides.

The cleavage of proteins in the methods of the present invention can be performed using both chemical and enzymatic methods.

Chemical cleavage methods include the use of cleaving agents such as, but not limited to, BNPS-skatole [2-(2-nitrophenylsulfenyl)-3-methylindole], CNBr, formic acid, hydroxylamine (NH₂OH) and iodosobenzoic acid, and NTCB+Ni (2-nitro-5-thiocyanobenzoic acid).

Enzymatic cleavage methods include digestion with enzymatic cleaving agents such as, but not limited to, Asp-N Endopeptidase, Arg-C Endopeptidase, Caspase 1, 2, 3, 4, 5, 6, 7, 8, 9, or 10, Chymotrypsin, Clostripain, Enterokinase, Factor Xa, Glutamyl Endopeptidase, Granzyme B, LysC Lysyl endopeptidase, Pepsin, Proline-Endopeptidase, Proteinase K, Staphylococal peptidase I, Thermolysin Thrombin, Trypsin. Parameters such as incubation time, enzyme/substrate ratio, pH and buffer can influence the specificity of certain proteases.

For the purpose of the present invention, typically cleavage methods and/or agents are chosen, which are specific and have a high efficiency. As explained in detail further on, the methods of the present invention typically rely on the comparison of experimental cleavage data with in silico cleavage data. It is therefore of importance that the theoretical cleavage pattern of a sample matches as much as possible the experimental data. For example, use of CNBr for cleaving C-terminally of Methionine can also result in the cleavage C-terminally of Tryptophane. Chymotrypsin which cleaves preferentially C-terminally of aromatic amino acids will also cleave C-terminally of other hydrophobic amino acids, depending from the incubation time and the concentration of enzyme in the sample.

Also the average size of the generated peptides is of importance. The shorter the peptides, the greater the chance that peptides from different proteins will have the same mass and even have the same sequence and will behave in an identical way in purification and analysis method. Accordingly, depending on the nature and complexity of the sample; an enzyme with a less commonly occurring cleavage site may be preferred.

According to a particular embodiment, the cleavage step in the methods of the present invention is performed with trypsin, in view of its high specificity and efficiency. Alternatively, where cleavage at both Lys and Arg results in too short peptides, other enzymes can be used such as endoproteinase Arg-C (Arginine specific), endoproteinase Lys-C (Lysine specific), S. aureus V8 protease (Asp/Glu specific). Alternatively, side chains of Lysine are modified by acetylation to limit tryptic cleavage to Arginine residues (and cysteine which is modified into homoarginine and becomes a substrate for trypsin).

In a further step of the methods of the present invention the complexity of the sample is reduced by isolating C-terminal peptides.

The cleavage of proteins into peptides in the cleavage step described above has the disadvantage that the high number of proteins potentially present in a sample is converted in an even much higher number of peptides, which in principle, all need to be analysed to identify all of the proteins present in the sample and potential protein processing having occurred thereon. In this way, redundant information is obtained, as many peptides of a same protein are analysed. Different methods have been described to reduce the complexity of a peptide sample. For instance, only peptides comprising a Cysteine can be isolated using a labelling reagent that is reactive against the thiol group of reduced cysteine and that carries a tag to isolate the labelled cysteine comprising peptide. However, some proteins have no Cysteine at all, while others have more than one Cysteine. Cysteine-labelling can thus only to a limited extent reduce the complexity of a sample to one peptide per protein without loosing information.

According to the present invention, the reduction of the complexity of the one or more samples to one peptide per protein is achieved by selecting the C-terminal peptides from a mixture of cleaved proteins. The selection of C-terminal peptides has certain advantages. The N-terminus is more prone to in vivo proteolytic processing than the C-terminus, which makes it difficult to predict which N-terminal peptides will be present in a cleaved protein sample. Additionally, many different modifications of the N-terminus exist either in vivo or as a result of the manipulation of a protein sample, such as by acetylation, formylation, and modification into pyroglutamic acid. Despite prediction methods on N-terminal Methionine processing (“N-end rule”) it is not always to predict the genuine N-terminal amino acid of a protein. Furthermore, the N terminus often contains signal sequences (such as for transmembrane transport sequences), which are conserved and make the sequences of N-terminal peptides less informative than those of C-termini. Also, differences in protein sequences due to alternative splicing occur more often at the N-terminal part of a protein than at the C-terminal part.

Accordingly, the methods of the present invention comprise the step selecting the C-terminal peptides of the cleaved proteins in the sample(s). Upon cleavage of a modified protein, the N-terminal peptide and all internal peptides of that protein obtain a new carboxylgroup, while the carboxyl groups of the original protein was modified in the modification step prior to the cleavage. The newly generated carboxyl groups are used for removal of the N-terminal and internal peptides from the mixture, either by binding these peptides directly to a matrix through the carboxyl group or by reacting the carboxyl group with an affinity label followed by isolation of the affinity tagged peptides on a affinity matrix. Methods for isolating C-terminal peptides are described, for example, in U.S. Pat. No. 6,156,527 (Schmidt), US2006/134724 (Fisher), and US2002/0106700 (Foote). N-terminal peptides and internal peptides can be reversibly bound via the carboxyl groups on ion exchangers, exploiting the difference in charge with the modified C-terminal peptides. Alternatively, the N-terminal and internal peptides are bound to a matrix functionalised with a carboxyl reactive group such as those described in the context of the carboxyl modification step (first method step) of the present invention, above. Another alternative embodiment of the isolation of C-terminal peptides in the methods of the present invention involves the covalent or non-covalent binding of an affinity tag to the carboxylgroup of the N-terminal and internal peptides. Suitable affinity tags include, but are not limited to, d-biotin or structurally modified biotin-based reagents, 1.2-diols, haptens such as dinitrophenyl or ligands which bind to a transition metal, such as the hexahistidine, or glutathione.

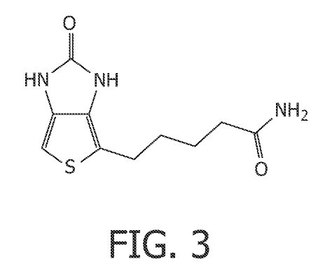

In a particular embodiment of the methods of the present invention, the reagent carbodiimide EDC is used to react a biotin molecule comprising a NH₂ group (such as for example depicted in FIG. 3) with the carboxylgroup of the internal and N-terminal peptides.

In a further step of the methods of the present invention, the isolated C-terminal peptides of one sample or two or more pooled samples are subjected to one or more peptide separation techniques.

Suitable separation techniques, which allow the separation of a complex peptide sample into multiple fractions, are known to the skilled person and include, but are not limited to isoelectric focusing, anion or cation exchange chromatography, reversed-phase HPLC, ion pair reversed-phase chromatography, affinity chromatography, . . . etc. Though suitable in principle, techniques such as SDS PAGE, 2-dimensional gel electrophoresis, size-exclusion chromatography are less appropriate for the separation of C-terminal peptides of generally limited length, as are those isolated in the methods of the present invention.

Several technologies to separate peptide digests by liquid chromatography have been described, including reversed-phase (RP)-HPLC, and 2-dimensional liquid chromatography. For peptide samples obtained from proteolytic digestions, 2D-LC approaches are particularly suitable for separation, providing also significant advantages with regard to automation and throughput. Also capillary electrophoresis (CE) is a method suitable for the separation of peptides.

2D-LC generally uses ion-exchange columns (usually, strong cation exchange, SCX) on-line coupled with a reversed phase column, operated in a series of cycles. In each cycle the salt concentration is increased in the ion-exchange column, in order to elute peptides according to their ionic charge into the reversed phase system. Herein, the peptides are separated on hydrophobicity by e.g. a gradient with CH₃CN.

Many parameters influence the resolution power and subsequently the number of proteins that can be displayed by LC-MS. Usually, the ‘on-line’ configuration between the first-dimension separation technique (SCX) and the second-dimension RP-HPLC separation approach is set up for sample fractionation. Ion exchange chromatography can be performed by stepwise elution with increasing salt concentration or by a gradient of salt. Typically, SCX is performed in the presence of, e.g. up to 30% acetonitrile, to minimize hydrophobic interactions during SCX chromatography. Prior to Reversed Phase chromatography on e.g. a C18 column, organic solvents such as acetonitrile are removed, or strongly reduced by e.g. evaporation.

As detailed above, the methods of the present invention can be performed either on individual samples, or can be used in the simultaneous analysis of two or more protein samples to avoid the variability introduced by the different processing steps, more particularly by the peptide separation methods described above. To discriminate between identical peptides originating from different samples, different options are envisaged.

According to a first embodiment of the methods of the present invention, the modification of the carboxylgroup of the intact protein in the first step of the invention is used as a differential labelling step, by reacting the carboxyterminus of the protein(s) with a detectable label. Once the differential labelling of the proteins of the different samples is performed, the samples can be pooled and further processing occurs on the pooled sample. Alternatively, the samples can be processed individually and pooled prior to analysis. However, in comparative proteomic analysis of two or more cleaved differential labelled protein samples, samples are ideally pooled as early as possible in the procedure to limit the variability between samples introduced by peptide separation techniques. The differentially labelled versions of a same peptide are then analysed together on MS to accurately compare the concentration of the individual peptides between the different samples.

Different labels can be used to discriminate peptides with the same amino acid sequence. However, in order to facilitate the identification of corresponding peptides after separation and MS analysis and to avoid having to generate complex databases, it is of interest to use labels which are identical in chemical structure such that differentially labelled peptides will behave similarly in chromatographic separation systems while generating a differential signal in MS.

In particular embodiments of the methods of the present invention, the different protein samples are labelled with isotopic labels. Isotopic labels have an identical chemical structure, such that the isotopically labelled identical peptides behave essentially identically in protein purification systems, but behave differentially on MS.

Examples of suitable isotopic labelling agents include the labels of so-called ICAT reagents as described in Gygi et al. (1999) Nat. Biotechnol. 17, 994-999 and U.S. Pat. No. 6,852,544. At present two different labelling reagents are commercially available which are SH reactive and contain a biotin affinity tag. U.S. Pat. No. 6,852,544 discloses combinations of COOH reactive groups, linked to isotopic labelling groups, which are suitable for labelling uncleaved proteins at the COOH terminus. An affinity tag such as the biotin tag is not needed in the present invention. The selection of peptides is performed on the carboxylgroup of the N-terminal and internal peptides.

Alternatively, the differential labelling of the protein samples can be ensured concomitantly with the modification step. According to this embodiment, the reagents used for the modification of carboxyl groups as described above, comprising one or more isotopes such as ²H, ¹³C, ¹⁵N, ¹⁷O, ¹⁸O or ³⁴S are also suitable for isotopic labelling. Examples include methylamine and methylamine-(d3) or ethylamine and ethylamine-(d5).

According to a further embodiment, the differential labelling of the protein samples in the methods of the present invention is performed as a labelling on the newly generated aminoterminus of the C-terminal peptides generated upon cleavage. To avoid simultaneous labelling on (internal) Lysine, the proteins can be modified with e.g. alkylating agents such as acetic anhydride prior to cleavage. According to this embodiment, differential labelling of the samples is performed after the cleavage step and optionally after the isolation of the C-terminal peptides.

Labelling groups which are suitable for N-terminal labelling include 2-tert-butyloxy-carbonylamino-2-phenylacetonitrile [BOC—ON]-(d0) or -(d9) acetyl chloride-(d0) or -(d3), benzoyl chloride-(d0) or (d5) or acetic anhydride-(d0) or -(d6).

Equally all NH₂ reactive ICAT labelling reagent disclosed in U.S. Pat. No. 6,852,544, either with, but normally without affinity label are suitable for isotopic labelling of the C-terminal peptides isolated in the present invention.

The labelling of the N-terminus of C-terminal peptides can be performed before or after the isolation of the C-terminal peptides, since it does not interfere with the purification, which is based on the C-terminus of the N-terminal and internal peptides. However, when the labelling is performed prior to the isolation no carboxyl groups may be present in the labelling reagent.

The methods of the present invention comprise an identification step which is based on comparing data on the physicochemical characteristics of the peptides with those of a database of peptides.

Accordingly, for each peptide fraction obtained in the one or more separation steps of the methods of the present invention, data are collected and stored relating to the behaviour of the peptide in the one or more separation steps, e.g. during chromatography. Such data include for instance the pH at which the purification was performed, the percentage of organic solvent at which a peptide elutes from a reversed phase column, the salt concentration at a given pH at which a peptide elutes from an ion exchange matrix, the binding (or not binding) of the peptide to a certain resin at a given pH etc.

Additionally or alternatively, further data can be collected for each peptide, which is not directly obtained from the peptide separation and purification step(s) in the methods of the present invention. Accordingly, for each peptide, a fraction of the isolated peptide can be stored to perform assays to determine properties which are not determined during purification. Such assays for example include, but are not limited to determination of the solubility, partition coefficient in water/organic solvent systems, detection of specific amino acids side groups (e.g. —OH, —SH, —NH₂).

In a further step of the present invention, the C-terminal peptide fractions which have been isolated as described above, are analysed by Mass Spectrometry. Where two or more samples are analysed simultaneously, the peptide fraction will potentially contain identical C-terminal peptides, which are differentially labelled. Alternatively, where the relevant protein has undergone proteolytic in vivo processing in one or more of the samples, the C-terminal peptides corresponding to the same parent protein may elute in different fractions which are analysed separately on MS.

The identification of C-terminal peptides corresponding to the parent proteins present in a sample in MS spectra is achieved by the high mass accuracy of high-resolution mass spectrometers. Mass measurements by spectrometry are performed by the ionisation of analytes into the gas phase. The mass-to-charge ratio (m/z) of the ionised molecules is determined and the number of ions for each individual m/z value is counted. Each feature in an MS spectrum is thus defined by two values, m/z and a measure on the number of ions detected.

In a further step of the methods of the present invention, the experimental determined mass of a C-terminal peptide is compared with the masses of in silico generated peptides in a database.

The mass of a peptide is correlated with its amino acid composition. Based on the mass alone however, it is not always possible to positively identify a peptide. For instance, mass alone will not allow to discriminate between peptides having the same amino acid composition but a different sequence (A1-A2-A3-A4-A5 versus A5-A1-A2-A3-A4). Furthermore certain masses can correspond to a set of peptides having a different sequence. For example a short peptide with amino acids with longer side chains can have the same mass as a longer peptide which has amino acids with shorter side chains.

When no selection is performed on the peptides of cleaved proteins, the mass of a tryptic peptide is correlated with all the masses of an in silico digest of the proteome of a certain organism.

Using the C-terminal peptide isolation as described in the present invention, the number of peptides obtained from a protein sample is strongly reduced. Accordingly, the in silico tryptic peptide database needs to contain only C-terminal peptides (so called C-terminal database).

Existing protein and sequence databases can be used as a basis to generate a database corresponding to the proteome of any organism. For an ever-increasing list of organisms, the complete genome, and the proteome deduced therefrom is known (www.ncbi.nlm.nih.gov/genomes). Thus, in silico peptide databases can be generated wherein protein cleavage and peptide isolation is simulated. Depending on the efficiency of a cleaving agent, the database can contain peptides wherein the cleavage is incomplete.

In a C-terminal database suitable in the context of the present invention, each entry includes the name of the parent protein and the mass of the corresponding C-terminal peptide. For each entry, also the amino acid composition is important, to calculate mass differences caused by natural post-translational modifications (e.g. phosphorylation on Serine, Threonine and Tyrosine), treatment of the sample (e.g. deamidation of Asparagine and Glutamine) or modifications introduced during the modification/labelling of the protein and isolation of the C-terminal peptides. For each proteolytic enzyme, a single primary database comprising the masses of unmodified C-terminal peptides is used to recalculate the mass depending on the type of modifications introduced before and after cleavage. In addition, potential post-translational modifications, which may or may not be present, are also taken in account by calculating the mass of peptides in the database. Additionally, where the use of labels is envisaged, the influence of the label on the mass of each of the peptides can be incorporated.

Nevertheless, as shown in U.S. Pat. No. 6,846,679 for a small set of in Influenzae proteins, the mass of an experimental C-terminal peptide can correspond to different peptides in the corresponding C-terminal database. Such database is thus not sufficiently informative to identify the parent protein of a C-terminal peptide solely on the mass of that peptide. Accordingly, the present invention provides for an identification of the C-terminal peptides based on not only m/z ratio, but including additional characteristics such as length (number of amino acids), amino acid sequence, weight, hydrophobicity, isoelectric point, etc.

According to a particular embodiment of the invention, the database of C-terminal peptides corresponds to the proteome of a specific cleaving agent, and this for a given species, corresponding to the origin of the samples. Such a peptide database also includes annotated splice variants. The in silico peptide database used in the methods of the present invention, includes calculated characteristics of C-terminal peptides like length in amino acids, amino acid sequence, molecular weight, hydrophobicity, isoelectric point, etc.

As indicated above, it has to be taken into account that proteins coming from in vivo sources are often post-translationally modified, e.g., through acetyl groups, formyl groups, or pyroglutamic acid residues, all of which will have an influence on the determined m/z in a mass spectrum). Accordingly, in one embodiment of the present invention synthetic C-terminal peptides are used as reference standards to validate the in silico calculated peptide characteristics.

The information from the synthetic peptide libraries is used to facilitate the identification of the nature of mass spectrometry peptide peaks, thereby optionally obviating de novo sequencing. The identification will be based on measured characteristics like HPLC retention time, isoelectric point and mass spec m/z value compared to available information stored in the in silico peptide library.

Different types of physicochemical data are considered, which, in combination with the m/z data of the C-terminal peptides allow positive identification of the parent protein.

One type of data envisaged is data which are predicted from the sequence information and/or which can be measured during peptide purification steps and MS, such as isoelectric point, net charge at different pH values, retention time on RP HPLC, UV absorption at 214 and 280 nm, tendency to elute from ion exchange columns at given pH and salt concentrations, hydrophobicity, and hydrophilicity.

Hydrophobicity can be calculated for example by the algorithm of Bull and Breese. (1974) Arch. Biochem. Biophys. 161, 665-670. Isoelectric points can be calculated for example on www.expasy.ch/tools/pi_tool.html. Retention times on reverse phase columns are for example predicted according to the method of Krohkin et al. (2004)Mol. Cell. Proteomics 3, 908-919.

As indicated above, the database used in the context of the present invention additionally or alternatively comprises data obtained in additional experiments and not directly derived from peptide purification, such as, but not limited to data on solubility, partition over water/organic solvent two phase systems, assays for the detection of protein reactive groups (OH, NH₂, SH) [ionisation potential, dipole moment, hydrogen bonding capacity. and ion mobility in gas phase].

Accordingly, the methods of the present invention which provide an identification based on a comparison with an annotated C-terminal database, allow identification of the corresponding parent protein with increased accuracy.

Optionally, the C-terminal peptide database used in the context of the present invention further comprises information on expression patterns of the parent protein, etc., which further help to identify the parent protein. Where the parent proteins differ in amino acid sequence except from their terminal peptides, the corresponding entries in the annotated C-terminal peptide database will indicate C-terminal peptides with identical mass and identical physicochemical properties. The further annotation of entries of with details on possible differential expression of the parent proteins during development of the organism, or tissue specific expression, can nevertheless allow the assigning of the correct parent protein to the isolated C-terminal peptide. Indeed, depending on the origin of the protein sample, it may be possible to select from the different possible parent proteins, one which expression matches with that of the sample.

In the methods of the present invention, for each peptide the mass is calculated compared to the annotated C-terminal peptide database. Accordingly, those entries are selected that have a calculated mass which corresponds to the measured mass of the isolated peptide. Depending of the MS apparatus and the type of sample, comparison is performed with the monoisotopic mass or with the average mass.

When the monoisotopic mass is used, typically a measuring error of 0.1 mass units is included to select entries from the database. When the average mass is used, typically a measuring error of 1 Da is included to select entries from the database. When the measured mass corresponds with only one entry in the database, the parent protein is immediately identified.

When the measured mass corresponds to more than one entry in the database, all these entries are selected as a subset. A further identification is performed based on the comparison of the physicochemical parameters of the isolated peptides with those for the subset of entries in the database. Typically, those physicochemical parameters that can be directly derived from the peptide purification steps are considered first. According to a particular embodiment, at least three physiochemical characteristics are considered and identification is performed based on a “best fit” analysis. When only one additional parameter is considered, the parameter which is chosen largely depends on the discriminating power of that parameter within the set of peptides in the C-terminal database with the same mass. For example if the peptides in the C-terminal database have differing amounts of aromatic amino acids, the UV absorption at 214 and 280 nm can be used as a selection criterion. If in another example, in a set of 3 peptides in the database with the same m/z ratio, all of these have the same net charge, but the distribution of the charge is different (e.g. one peptide has no charged amino acids, another has one Arg and one Asp, and another has two Arg and two Asp), the behaviour on ion exchange can be used as a criterion to correlate the isolated peptide with one specific peptide in the subset of the database.

A further aspect of the present invention provides devices and instruments suitable for carrying out the methods of the present invention.

Prior to MS analysis, the methods of the present invention comprise a number of protein processing steps (protein modification, protein cleavage) and isolation and purification steps (C-terminal peptide isolation, separation of isolated peptides). Accordingly, the devices suitable for carrying out the methods of the present invention comprise appropriate reaction chambers, with corresponding sources of reagents (modification reagent, cleaving agent) and separation and isolation units (typically chromatography units). As appropriate separation of individual C-terminal peptides often require sequential separation techniques, the devices suitable for performing the methods of the present invention optionally contain or are connected to two or more suitable separation instruments, such as electrophoresis instruments, chromatography instruments, such as, but not limited to capillary electrophoresis (CE) instruments, reverse-phase (RP)-HPLC instruments, and/or 2-dimensional liquid chromatography instruments, . . . etc.

An essential feature of the methods of the present invention is the determination of the mass of the isolated C-terminal peptides. Accordingly, the devices for performing the methods of the present invention comprise a mass spectrometric instrument. A typical mass spectrometric instrument consists of 3 components, an ion source in order to vaporise the molecules of interest, a mass analyser, which determines the measures the mass-to-charge ratio (m/z) of the ionised molecules, and a sensor that registers and counts the number of ions for each individual m/z value. Each feature in an MS spectrum is defined by two values, m/z and a measure on the number of ions, which reached the detector of the instrument.

The ionisation of proteins or peptides for mass analysis in a spectrometer is usually performed by Electrospray ionisation (ESI) or matrix-assisted laser desorption/ionisation (MALDI). During the ESI process analytes are directly ionised out of solution and ESI is therefore often directly coupled to liquid-chromatographic separation tools (e.g., reversed phase HPLC). MALDI vaporises via laser pulses dry samples mixed with small organic molecules that absorb the laser energy like cinnamic acid to make the process more effective.

The mass analyser is a key component of the mass spectrometer; important parameters are sensitivity, resolution, and mass accuracy. There are five basic types of mass analysers currently used in proteomics. These include the ion trap, time-of-flight (TOF), quadrupole, Orbitrap, and Fourier transform ion cyclotron (FTICR-MS) analysers. Tandem MS or MS/MS can be performed in time (ion trap) and in place (with all hybrid instruments such as e.g. LTQ-FTICR, LTQ-Orbitrap, Q-TOF, TOF-TOF, triple quad and hybrid triple quadrupole/linear ion trap (QTRAP))

A particular embodiment of the invention relates to a device (100) for isolating and analysing C-terminal peptides protein samples comprising at least two sample sources (101), a modification/labelling unit (102), with corresponding modifying agents/label sources (103), a cleavage unit (104), a C-terminal peptide isolation unit (105), a peptide separation unit (106), a mass spectrometer unit (108) and a control circuitry and data analysis unit (109), connected to a read-out unit (110). The device can be configured to ensure pooling of the samples prior to the cleaving step (pooled sample enters cleavage unit) or after the cleaving step (samples pass through cleavage unit individually). In particular embodiments separation unit (106) comprises two consecutively linked separation systems (1106) and (2106), wherein the first separation system (1106) is e.g. a cation exchange chromatography system and separation system, and the second separation system (2106) is typically a HPLC reversed phase system. Mass spectrometer element (108) consists of a unit, which separates isotopic forms of peptides.

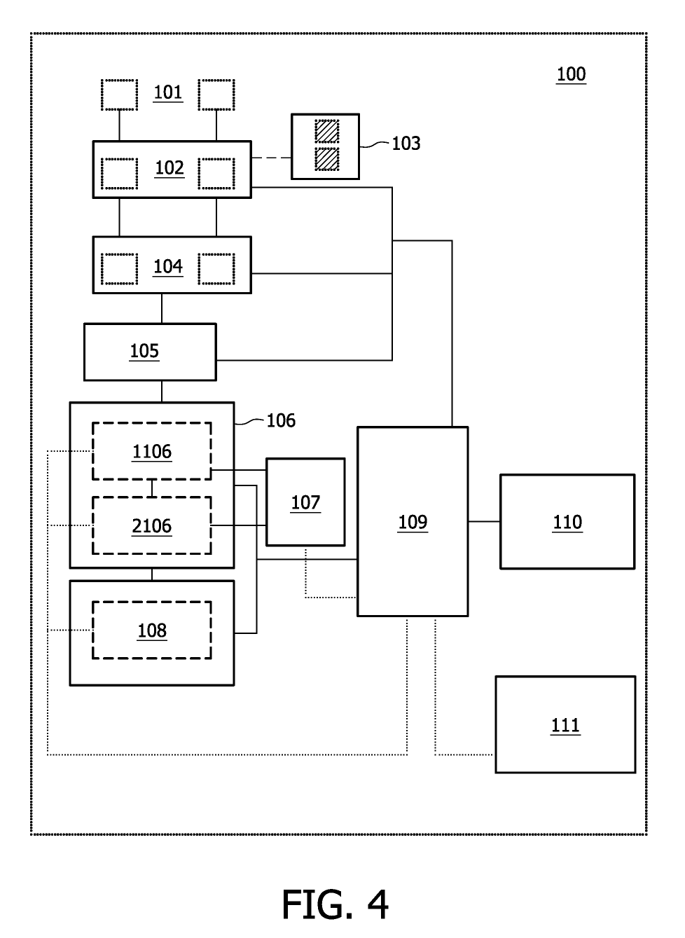

Particular embodiments of the device of the invention further comprise an analysis unit (107) wherein one or more physicochemical properties of a purified peptide are determined and/or registere. Data on the experimental mass of a peptide and its physicochemical properties obtained during purification and optionally obtained in the analysis unit are compared with an annotated database (111) of C-terminal peptides (indicated by dotted lines in FIG. 4).

### EXAMPLES

### Example 1

### Isolation of C-Terminal Peptides

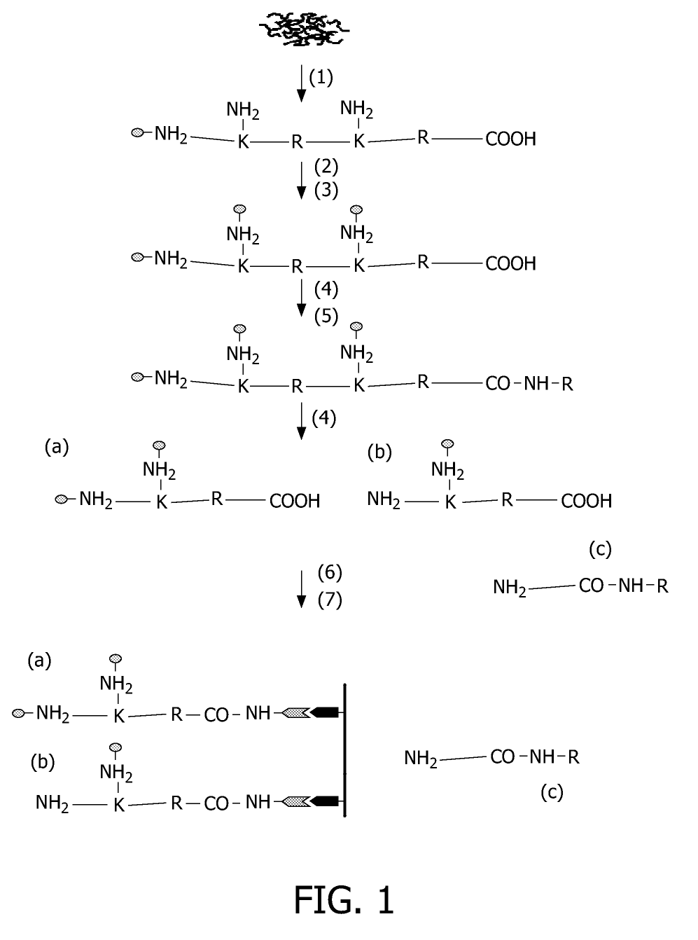

A flowchart of the isolation of C-terminal peptides is outlined in FIG. 1.

A protein extract is isolated from a tissue using standard methods. The side chains of Cysteine are alkylated and the amines at the N-terminus and the side chain of Lysine are acetylated. In a next step, the free carboxyl groups of the C-terminal amino acid (as well as the reactive carboxyl groups on Glutamic acid and Aspartic acid) are activated by 1-Ethyl-3-[3-dimethylamino-propyl]carbodiimide hydrochloride (EDC) or 1-ethyl-3(3-dimethyl-aminopropyl)-carbodiimide (EDAC) in accordance with the method as described in Grabarek & Gergely (1990) Anal Biochem. 185, 131-135.

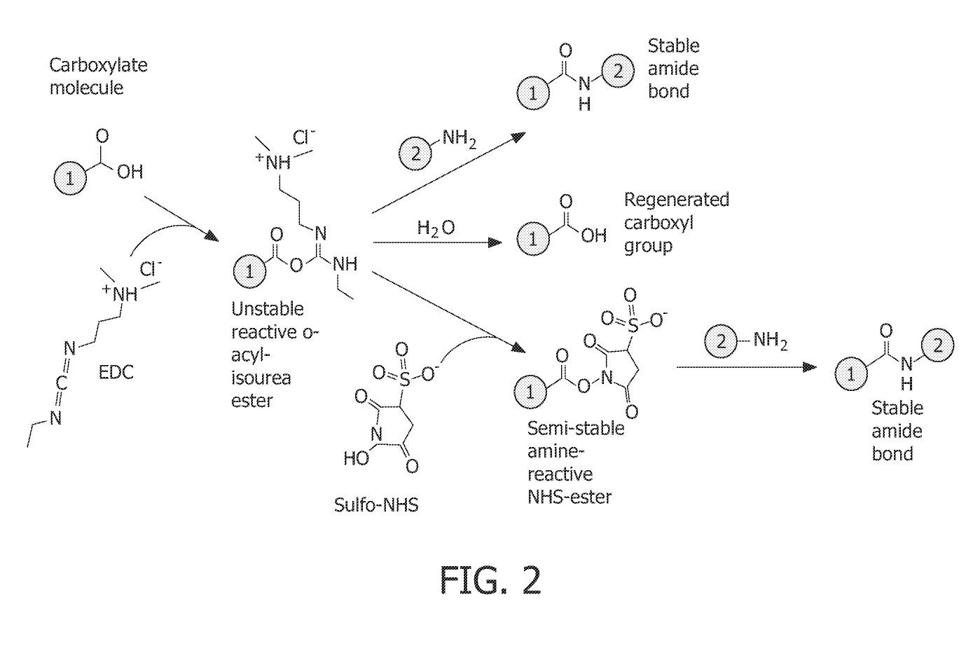

EDC reacts with a carboxyl group on a protein (molecule 1 in FIG. 2), forming an amine-reactive O-acylisourea intermediate. This intermediate may react with an amine on NH—R (molecule 2 in FIG. 2), yielding a conjugate of the two molecules joined by a stable amide bond. However, the intermediate is also susceptible to hydrolysis, making it unstable and short-lived in aqueous solution. The addition of Sulfo-NHS (5 mM) stabilises the amine-reactive intermediate by converting it to an amine-reactive Sulfo-NHS ester, thus increasing the efficiency of EDC-mediated coupling reactions. The amine-reactive Sulfo-NHS ester intermediate has sufficient stability to permit two-step cross-linking procedures, which allows the carboxyl groups on one protein to remain unaltered. The EDC-activated COOH group is coupled to an amino-group containing molecule, NH₂—R. NH₂—R can be a molecule, which improves the ionisation process of the C-terminal peptide by easily attracting a positive charge during this procedure. On the other hand, the reactive molecule must not contain any further carboxyl group. In the present example NH₂—R is ethylamine which can be isotopically labelled.

Subsequently, the protein sample is enzymatically digested with trypsin to generate a mixture of peptides.

N-terminal and internal peptides in the digest contain a free C-terminal amino acid, while C-terminal peptides have a modified carboxyl group by the above-described reaction.

The free C-terminal carboxyl groups of the internal and the N-terminal peptides are isolated via biotin affinity chromatography. This step leads to a separation of internal and N-terminal peptides and leaves the C-terminal peptides in the solution. The reaction is performed by the carbodiimide mediated reaction described above wherein R—NH₂ is a modified biotin as shown in FIG. 3.

All peptides except the C-terminal peptides of the peptide digest are removed from the solution by selective affinity depletion of these peptides. The very C-terminal peptides which are in the solution, are further fractionated by (multi)dimensional liquid chromatography followed by mass spectrometry analysis.

### Example 2

### Identification of Peptides with Similar Mass

The present example shows the need of supplementing mass data of peptides with additional parameters. From a motif search on Prosite (ScanProsite on www.expasy.org/prosite) a C-terminal tryptic peptide of 8 amino acids of a human protein with possible clinical relevance was chosen, namely the sequence SFPNIGSL of Exostosin 2 [SEQ ID. NO: 1].

The calculated average mass of SEQ ID. NO:1 (833.94) was used to identify with Profound (prowl.rockefeller.edu) peptides with a calculated mass within in 1 Da of the theoretical value. This was done by performing an in silico tryptic digest of human proteins allowing no partial cleavage and selecting a number of peptides which are C-terminal (see table 1).

TABLE 1
Mass and physiochemical parameters of C-terminal peptides with related mass.

| | | 2) | | 4) | 5) | 6) | |
|---|---|---|---|---|---|---|---|
| | 1) | sequence | 3) | arom. | phob. | phil. | 7) |
| | mass | [SEQ ID. NO:] | pI | (YFW) | (VILMYFW) | (STDEQN) | RT |
| Extostosin 2 | 833.94 | SFPNIGSL | 5.24 | 1 | 2 | 2 | 22.6 |
| | | [SEQ ID. NO: 1] | | | | | |
| intramembrane | 833.95 | SMSTQAPL | 5.24 | — | 2 | 4 | 18.6 |
| protease IMP5 | | [SEQ ID. NO: 2] | | | | | |
| HCN3 protein | 832.97 | GLQLSANM | 5.52 | — | 3 | 3 | 20.0 |
| | | [SEQ ID. NO: 3] | | | | | |
| FLJ00242 protein | 834.93 | FQALNSVG | 5.52 | 1 | 2 | 3 | 19.9 |
| | | [SEQ ID. NO: 4] | | | | | |
| biphenyl hydrolase- | 834.92 | LAEDFLQ | 3.67 | 1 | 3 | 3 | 22.5 |
| like protein | | [SEQ ID. NO: 5] | | | | | |

1, 3) average mass and pI are calculated on www.expasy.ch/tools/pi_tool.html
4) number of aromatic amino acids
5) number of hydrophobic amino acids
6) number of hydrophilic amino acids
7) retention time of peptides on reverse phase is calculated on http://hs2.proteome.ca/SSRCalc/SSRCalc.html (with parameters a = 10 and B = 0.48)

Typically peptides are separated by a combination of ion exchange chromatography and reversed phase HPLC. Using an ion exchange column wherein the salt concentration is increased, peptides elute according to their isoelectric point. Based on the pI of the above peptides, they will elute as three fractions (SEQ ID. NO: 1 and SEQ ID. NO: 2, SEQ ID. NO:3 and SEQ ID. NO:4 and SEQ ID. NO: 5) wherein the peptides with a pI closest to the pH of the buffer will elute first. Upon reversed phase chromatography SEQ ID NO: 1 and SEQ ID. NO:2 will elute at different positions since they have different amounts of hydrophilic and hydrophobic amino acids. It is also very easy to discriminate SEQ ID. NO:1 from SEQ ID. NO:2 and SEQ ID. NO: 3 from SEQ ID NO: 4 based on the UV absorption at 280 nm and 214 nm which are typically used for the detection of proteins on RP-HPLC. The peptides with SEQ ID. NO: 2 and 3 are easily recognised, as they will hardly absorb UV light at 280 nm.

1. A method for identifying a protein in a protein sample comprising the steps of:
a) modifying carboxyl groups of the proteins in the protein sample,
b) cleaving the proteins in the protein sample into peptides with a cleavage agent,
c) isolating from said peptides the C-terminal peptides from the N-terminal and internal peptides,
d) subjecting the isolated C-terminal peptides to one or more peptide purification steps, so as to obtain purified C-terminal peptides,
e) determining or calculating at least one physicochemical property, other than the mass, of the purified C-terminal peptides,
f) determining the mass of a purified C-terminal peptide on MS,
g) comparing the mass and the at least one other physicochemical property of the purified C-terminal peptide to a database comprising the mass and one or more physicochemical properties of all C-terminal peptides generated by said cleavage agent, so as to identify the parent protein of the purified C-terminal peptide.

2. The method of claim 1, wherein step (g) comprises identifying for each of the purified C-terminal peptides, one or more C-terminal peptides in the database with a mass corresponding to the purified C-terminal peptide, and, when more than one peptide are identified for one purified C-terminal peptide, comparing a physicochemical parameter of the purified C-terminal peptide with the more than one peptides identified in the database.

3. The method of claim 1, wherein the protein sample is from a species and the database comprises the mass and physicochemical properties of all C-terminal peptides of that species generated by said cleavage agent.

4. The method according to claim 1, wherein the protein is identified simultaneously in two or more samples and wherein the method comprises
in step (a) performing the modification with a set of differential labelling reagents,
an additional step of pooling the two or more samples prior to step (d),
prior to step (f) identifying the nature of the label so as to identify the sample from which the peptide originates,
in step (g) comparing the mass and the at least one other physicochemical property of the purified C-terminal peptide to a database comprising the mass of and physicochemical properties of all C-terminal peptides generated by said cleavage agent, so as to identify said C-terminal peptides.

5. The method according to claim 1, wherein the at least one physicochemical property is determined during the one or more peptide purification steps.

6. The method according to claim 1, wherein the at least one physicochemical property is selected from the group of pI, retention time during reversed phase chromatography and the ratio of UV absorption at 280 and 214 nm.

7. The method of claim 1 wherein the modification in step a) is performed using a carbodiimide reaction with primary amines.

8. The method of claim 1, wherein the isolation of C-terminal peptides in step (c) comprises the step of reacting the carboxylgroup of N-terminal and internal peptides via a carbodiimide mediated reaction with a modified biotin carrying a primary amine group.

9. A method for isolating C-terminal peptides from a protein sample comprising the steps of:
a) reacting carboxyl groups of intact proteins via a carbodiimide with primary amines,
b) cleaving the intact proteins with a cleavage agent into peptides,
c) reacting the carboxylgroup of N-terminal and internal peptides via a carbodiimide with an affinity tag carrying a primary amine group,
d) binding the tagged peptides to an affinity matrix and collecting the non-bound peptides, said unbound peptides being the non-tagged peptides thereby isolating from said peptides obtained under (b) the C-terminal peptides from the N-terminal and internal peptides.

10. The method according to claim 9, wherein the affinity tag is biotin.

11. A database of C-terminal peptides of proteins of an organism cleaved in silico by a cleaving agent wherein each peptide is characterised by
a protein identifier,
the amino acid composition,
the mass and,
one or more physicochemical properties.

12. The database according to claim 11, wherein the one or more physicochemical properties of said C-terminal peptides are selected from the group consisting of the calculated retention time on reverse phase chromatography, the net charge at a given pH, and the isoelectric point of said C-terminal peptides.

13. The database according to claim 11, wherein the organism is a human.

14. The database according to claim 11, wherein the cleaving agent is trypsin.

15. The database according to claim 11, wherein the peptides include C-terminal peptides resulting from an incomplete cleavage with said cleaving agent whereby one cleavage position is missed.

16. Use of a database according to claim 11 for the identification of proteins.

17. A device (100) for identifying proteins in one or more samples based on their C-terminal peptides, the device comprising at least one sample source (101), a modification/labelling unit (102), with at least one corresponding modifying agents/label source (103), a cleavage unit (104), a C-terminal peptide isolation unit (105), a peptide separation unit (106), an analysis unit (107) for determining and/or registering one or more physicochemical properties of a purified peptide, a mass spectrometer unit (108) a control circuitry and data analysis unit (109) and a connection to a database (111) comprising the masses of all C-terminal peptides of proteins cleaved in silico using a cleaving agent annotated with physicochemical properties of the C-terminal peptides.
